(19) United States
(12) Patent Application Publication
(10) Pub. No.: US 2018/0009949 A1
(43) Pub. Date: Jan. 11, 2018

(54) DINADIC PHENYL AMINE REACTIVE ENDCAPS

(71) Applicant: THE BOEING COMPANY, Chicago, IL (US)

(72) Inventors: Hyman Ralph LUBOWITZ, Rolling Hills Estates, CA (US); Thomas Karl TSOTSIS, Santa Ana, CA (US)

(21) Appl. No.: 15/653,655

(22) Filed: Jul. 19, 2017

Related U.S. Application Data
(63) Continuation of application No. 11/755,790, filed on May 31, 2007, now abandoned.

(51) Int. Cl. C08G 73/10; C07D 307/89; C07D 307/93; C08J 5/24; C07D 209/56

(52) U.S. Cl. CPC C08G 73/101; C08G 73/1014; C08J 5/24; C08G 73/1017; C07D 209/56; C07D 307/93; C07D 307/89; C08J 2379/08

(57) ABSTRACT

Dinadic phenyl amine reactive endcap monomers for application in high-temperature polymeric composites are described. The amine group of the endcap is directly reacted with a desired chemical backbone to provide the preferred rigidity and chemical resistance. The ability of the amine group to react with a wide variety of chemical backbones allows the tailoring of formulations for various application temperatures, mechanical properties, processes and resistances while retaining the high degree of crosslinking that yields excellent temperature stability, ease of processing and the necessary toughness. Polyimide oligomers comprising the reaction product of at least one dinadic phenyl amine endcap monomer and a chemical backbone, preferably with a molecular weight not exceeding about 1000-3000, suitable for high temperature composites are described. The dinadic phenyl amine endcaps may be reacted with an acid anhydride capped precursor to form polyimide resins suitable for high-temperature composites.

### CROSS-REFERENCE TO RELATED APPLICATIONS

This application is a continuation of U.S. patent application Ser. No. 11/755,790 filed on May 31, 2007. The disclosure of the above application is incorporated herein by reference.

### BACKGROUND OF THE INVENTION

Embodiments of the present invention relate to amine functional dinadic phenyl reactive endcaps and polyimide oligomers having high thermal and oxidative stability and improved mechanical properties. Additionally, embodiments of the invention pertain to high performance polymeric composites comprising dinadic phenyl amine endcaps.

In general, the use of multifunctional endcaps is well known in the art. For example, U.S. Pat. No. 4,536,559 discloses a series of thermoplastic resins that resist attack by organic solvents because they include di-imidophenol endcap monomers to provide crosslinking. The use of multifunctional endcaps in epoxy-based composites is well known in the art. However, such epoxy-based composites are wholly unsuitable for high-temperature applications.

Nadic endcaps were first described for use in preparing polyimide composites in U.S. Pat. No. 3,565,549 and later in U.S. Pat. No. 3,745,149 as well as others. Dinadic endcaps with acid-chloride functionality are by U.S. Pat. Nos. 5,227,461 and 4,935,523, among others as well as by Soutchcott, et al. It is desirable to have this dinadic feature with different functionality than disclosed by previous workers to enable the application of such endcaps to a greater variety of backbones. Accordingly, a dinadic endcap with amine functionality is of benefit because it provides this feature allowing all backbones with terminal functionalities that can react with amines to be used. This enables dinadic endcaps to be used with a much wider variety of backbone chemistries than possible with existing methods.

### BRIEF SUMMARY OF THE INVENTION

Embodiments of the invention precisely meet the aforementioned needs by providing dinadic phenyl amine endcap monomers; wherein the endcaps can be reacted with a chemical backbone of moderate molecular weight to form polyimide oligomers suitable for high temperature composites. The amine functionality of the present endcap enables use of a wide variety of backbones. Accordingly, formulations can be specifically prepared, without sacrificing crosslink density, to address diverse applications requiring stability at various temperatures, application-specific mechanical properties, as well as different chemical resistances. Due to the difunctionality of the endcaps and the use of moderate to low molecular weight backbones, polyimide oligomer embodiments of the invention are capable of providing an increased degree of crosslinking. Polyimide oligomer embodiments of the invention are easily processed and exhibit excellent temperature stability and toughness. Therefore, polymeric resins comprising dinadic phenyl amine endcaps are ideal for high performance composites as presently needed by the aerospace industry.

### DETAILED DESCRIPTION OF THE INVENTION

The present invention now will be described more fully hereinafter, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements

In one of its aspects, embodiments of the present invention relate to dinadic phenyl amine reactive endcap monomers for application in high-temperature polymeric composites. When a molecule is terminated with endcaps of various embodiments of the present invention, it acquires tetra-functionality that promotes a greater degree of crosslinking and polymer-network toughness. The amine group is known to be quite reactive with a wide variety of chemical groups. Thus, the dinadic phenyl amine functional endcaps are easily reacted with a wide variety of chemical backbones that have been suitably functionalized to form desirable oligomers for high-temperature polymeric composites. Specifically, the amine group of the endcap is used to react with a desired chemical backbone to provide the desired rigidity and chemical resistance. The ability of the amine group of the endcap to react with a wide variety of chemical backbones allows the tailoring of formulations for various application temperatures, mechanical properties, processes and resistances while retaining the high degree of crosslinking that yields excellent temperature stability, ease of processing and the necessary toughness.

In one embodiment, a dinadic phenyl amine endcap for application in high temperature polymeric composites may be selected from the formulae:

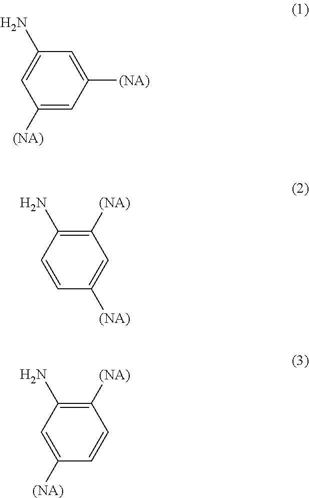

wherein (NA) is nadic anhydride illustrated by the formula:

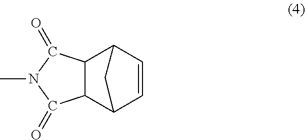

Amines can be obtained by numerous methods known in the art including direct amination, arylation or alkylation of ammonia or amines and free radical addition of amines to olefins. Embodiments of the dinadic phenyl amine reactive endcaps of the present invention can be readily synthesized by first converting 3,5-diamino benzoic acid to a dinadic carboxylic acid as follows:

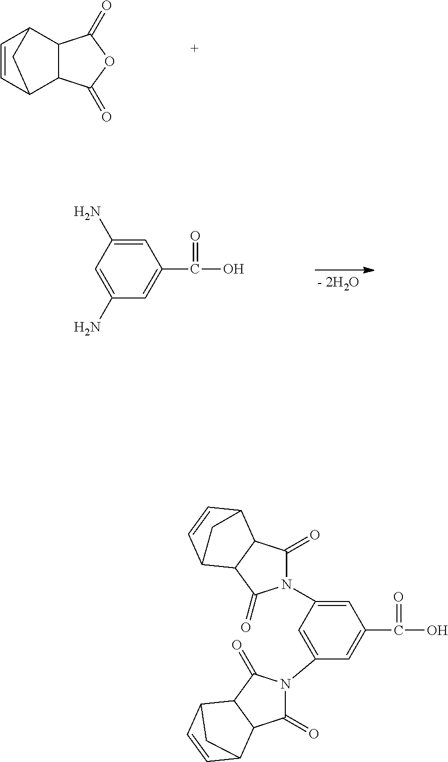 [SMILES: O=C1OC(=O)C2C3C=CC(C3)C12 ; Nc1cc(N)cc(C(=O)O)c1 ; O=C(O)c1cc(N2C(=O)C3C4C=CC(C4)C3C2=O)cc(N2C(=O)C3C4C=CC(C4)C3C2=O)c1]

In one embodiment, the dinadic carboxylic acid is reacted with sodium azide to form a cyanate, which is then converted to an amine by hydrolysis via a Curtius rearrangement as illustrated below.

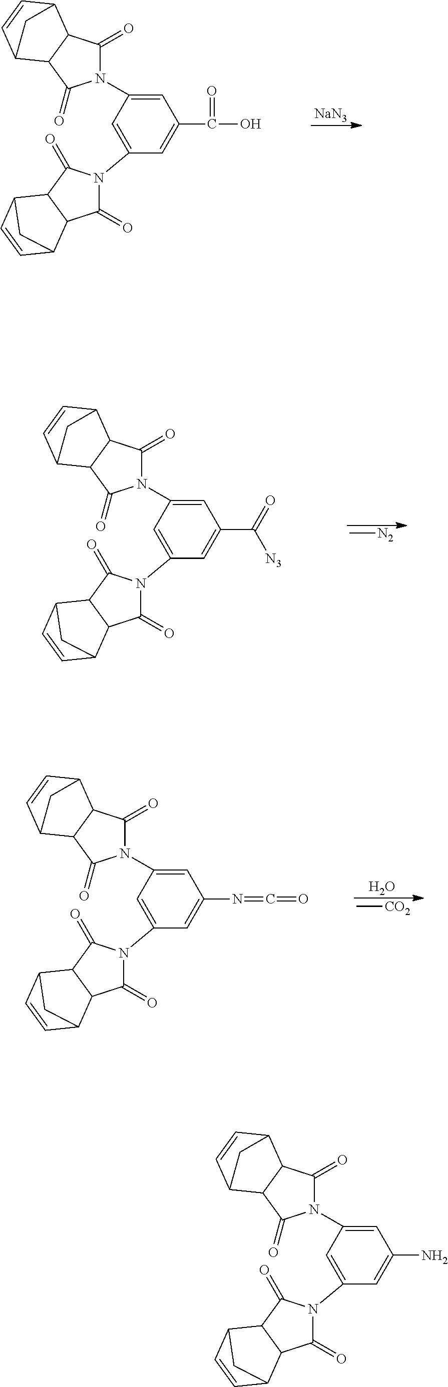

Alternatively, the dinadic carboxylic acid may be reacted with an azide under acidic conditions to form an acyl azide, which rearranges to an isocyante. The isocyanate is hydrolyzed to carbamic acid and decarboxylated to form the amine as illustrated below.

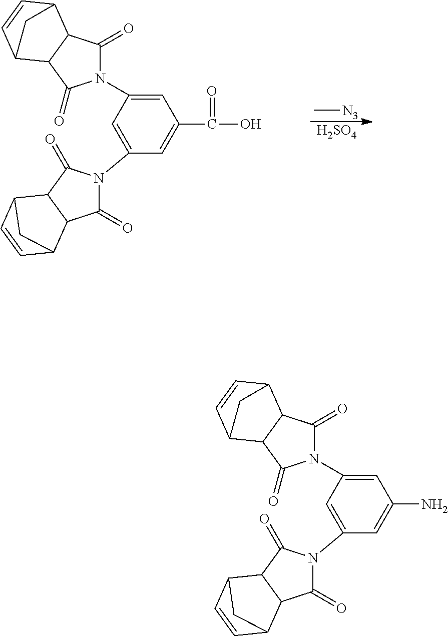

In one alternative embodiment, dinadic phenyl amine reactive endcaps are synthesized by first converting 2,4-diamino phenol to form a dinadic phenol as follows:

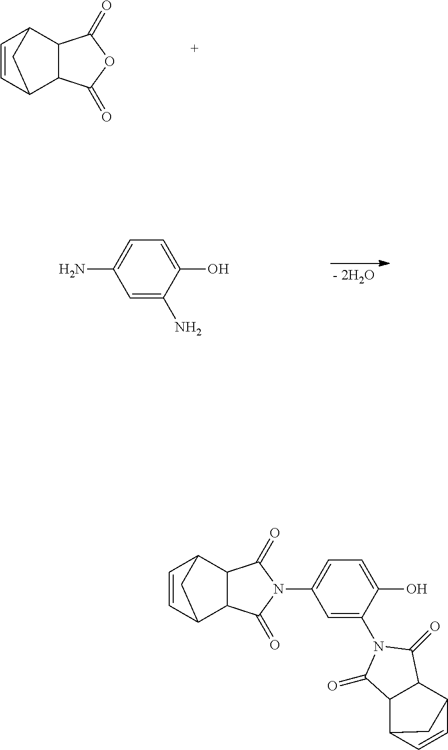 [SMILES: O=C1OC(=O)C2C3C=CC(C3)C12 ; Nc1ccc(O)c(N)c1 ; O=C1C2C3C=CC(C3)C2C(=O)N1c1ccc(O)c(N2C(=O)C3C4C=CC(C4)C3C2=O)c1]

The dinadic phenol above can be converted to a dinadic phenyl amine by utilizing either a Curtius rearrangement or a Schmidt reaction.

In another aspect, the invention pertains to polyimide oligomers comprising the reaction product of at least one dinadic phenyl amine reactive endcap monomer and any chemical backbone capable of reacting with an amine and is suitable for high-temperature composites. Since the amine group on the dinadic phenyl amine endcap can react with numerous functional groups, endcap embodiments of the present invention can be reacted with chemical backbones having a wide variety of functionality. In one preferable embodiment, the dinadic phenyl amine endcaps are reacted with an acid anhydride capped precursor to form polyimide resins suitable for high-temperature composites.

Furthermore, the dinadic capped oligomers may comprise low to moderate molecular weight precursors. For example, in various embodiments precursors should preferably not exceed a molecular weight of about 2500 to about 5000. While in other embodiments, the precursors should preferably not exceed a molecular weight of about 1000 to about 3000. By using precursors with such molecular weights, several benefits are readily realized. In particular, increased crosslink density is obtained due to the increased percentage of reactive sites, namely the dinadic functional groups of the endcaps, relative to the backbone. Additionally, the use of lower molecular weight precursors improves the material's melt-processability.

In one embodiment, polyimide oligomers of the present invention comprise the reaction of at least one dinadic phenyl amine endcap monomer and a chemical backbone according to the formula:

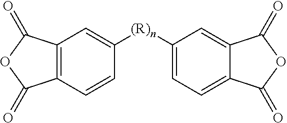

wherein R is selected from the group consisting of

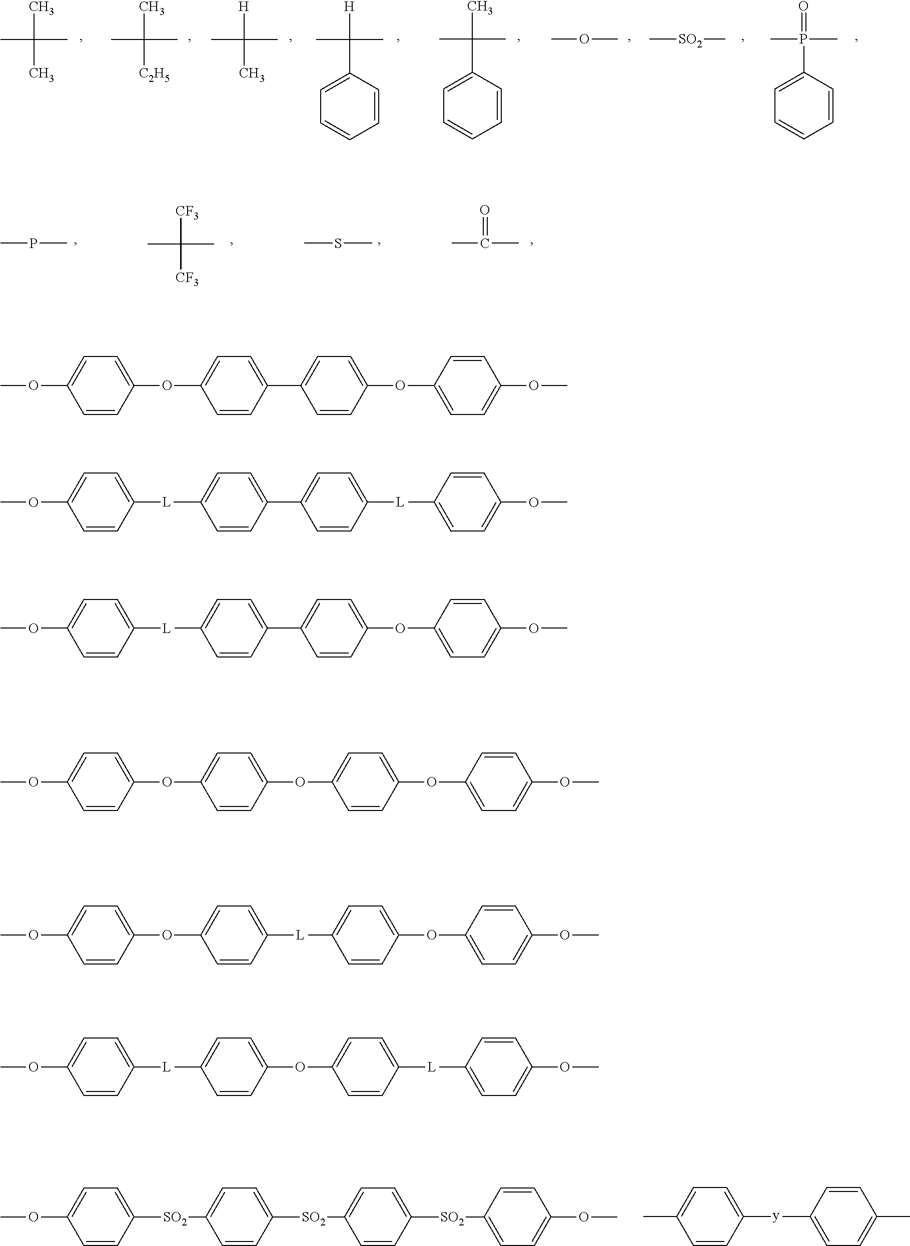

wherein L is —CH₂—, —(CH₃)₂C—, —O—, —S—, —SO₂—, or —CO—;
wherein y is —SO₂—, —S—, —(CF₃)₂C—, —O—, or —(CH₃)₂C—;
and in certain embodiments n is selected such that the molecular weight does not exceed about 3000.

In another embodiment, polyimide oligomers of the present invention comprise the reaction product of at least one dinadic phenyl amine reactive endcap monomer and a chemical backbone according to the formula:

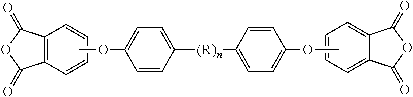

wherein R is selected from the group consisting of

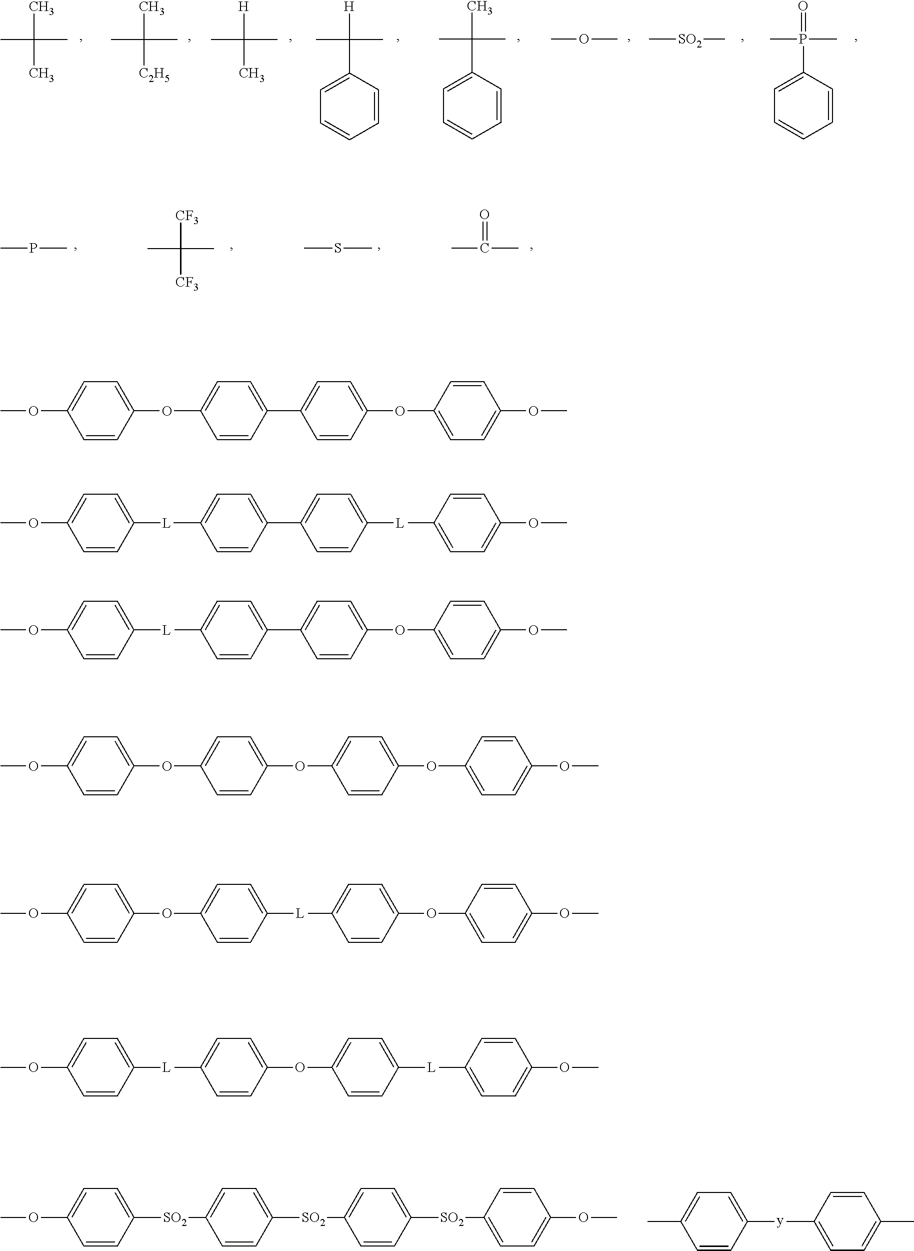

wherein L is —CH₂—, —(CH₃)₂C—, —O—, —S—, —SO₂—, or —CO—;
wherein y is —SO₂—, —S—, —(CF₃)₂C—, —O—, or —(CH₃)₂C—;
and in certain embodiments n is selected such that the molecular weight does not exceed about 3000.

In addition to the traditional schemes for synthesizing polyimide oligomers, various polyimide oligomer embodiments of the of the present invention can be formed by directly reacting at least one dinadic phenyl amine endcap with any chemical backbone that is capable of reacting with an amine and is suitable for high-temperature composites. In various embodiments, at least one dinadic phenyl amine endcap is directly reacted with a precursor capped with acid anhydrides to form an oligomer which is suitable for high-temperature composites. The direct reaction between a dinadic amine endcap and an acid anhydride capped precursor forms a tetra-functional oligomer appropriate for high-temperature composites. Accordingly, embodiments of the present invention provide a method of synthesizing a dinadic capped oligomer suitable for high temperature compositions whereby costly intermediate steps are eliminated.

In one embodiment, an oligomer according to the present invention may be formed as follows:

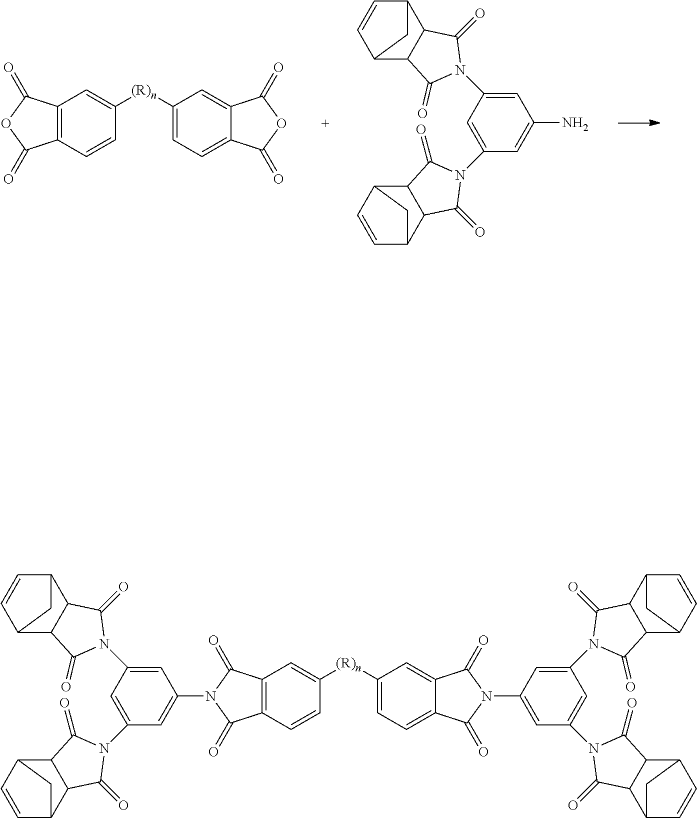

wherein R is selected from the group consisting of

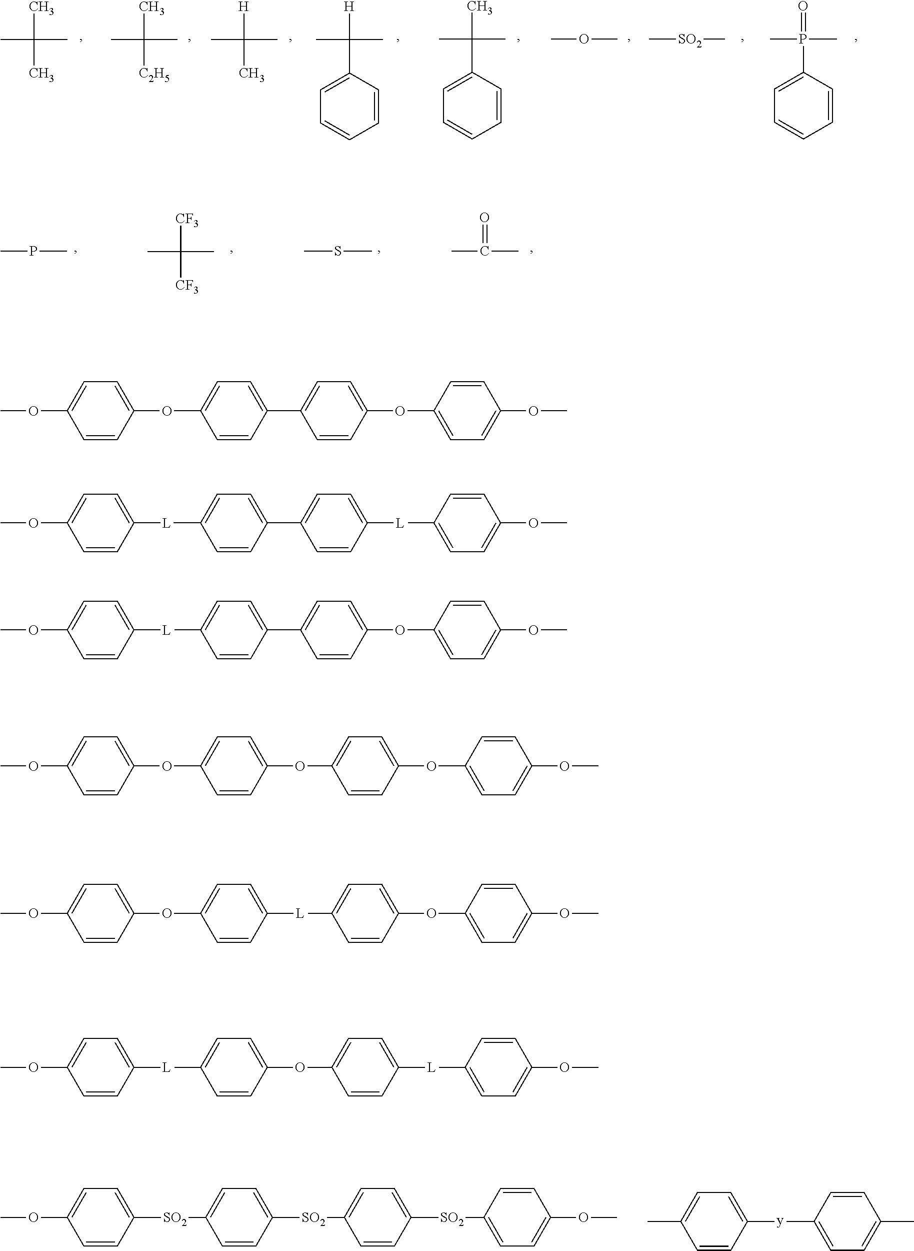

wherein L is —CH₂—, —(CH₃)₂C—, —O—, —S—, —SO₂—, or —CO—;
wherein y is —SO₂—, —S—, —(CF₃)₂C—, —O—, or —(CH₃)₂C—;
and in certain embodiments n is selected such that the molecular weight does not exceed about 3000.

In yet another aspect, the present invention is directed to composites or prepregs including polymeric resin comprising at least one dinadic phenyl amine endcap. Embodiments of the present invention, having a density less than metal counterparts, are ideal for replacing metallic structures to reduce weight. Where high-temperature strength also drives the design, a material with higher allowable strength at elevated temperatures, such as embodiments of polymeric composites including resin comprising at least one dinadic phenyl amine endcap, will reduce overall structural weight. Implementation of the dinadic phenyl amine endcap embodiments of the present invention into polymer formulations will allow for the production of lighter-weight composite structures to be used in place of metallic structures on aerospace or similarly demanding vehicles. Thus, reducing the overall weight of aerospace vehicles or the like. Also, polymeric composites according to embodiments of the present invention can be used to replace other high-temperature composites that require a thermal-protection layer. Similarly, this too will reduce the weight of aerospace vehicles by obviating the need for the thermal protection. Although advantageous for use with aerospace vehicles, other applications, such as other weight sensitive applications, may also employ polymeric composites according to embodiments of the present invention.

Composites and prepregs comprising polymeric resin including at least one dinadic phenyl amine endcap can by prepared by any conventional technique known in the art. For example, in certain embodiments the melt viscosity of the dinadic phenyl amine endcapped oligomers exhibit a melt viscosity such that a composite can be prepared by known liquid molding techniques such as resin-transfer molding and resin film infusion among others. Depending on the application, the reinforcement materials can include, for example, woven fabrics, continuous or discontinuous fibers (in chopped or whisker form), ceramics, organics, carbon (graphite), or glass.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A composite comprising:
polyimide oligomer of Formula: 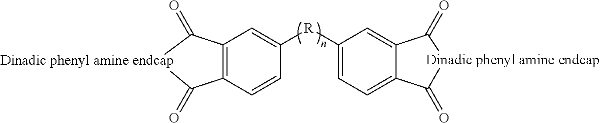
wherein:
each dinadic phenyl amine endcap is selected from the group consisting of : 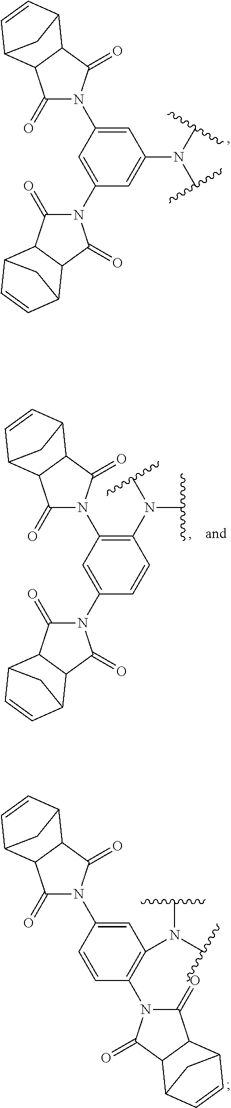
R is selected from the group consisting of 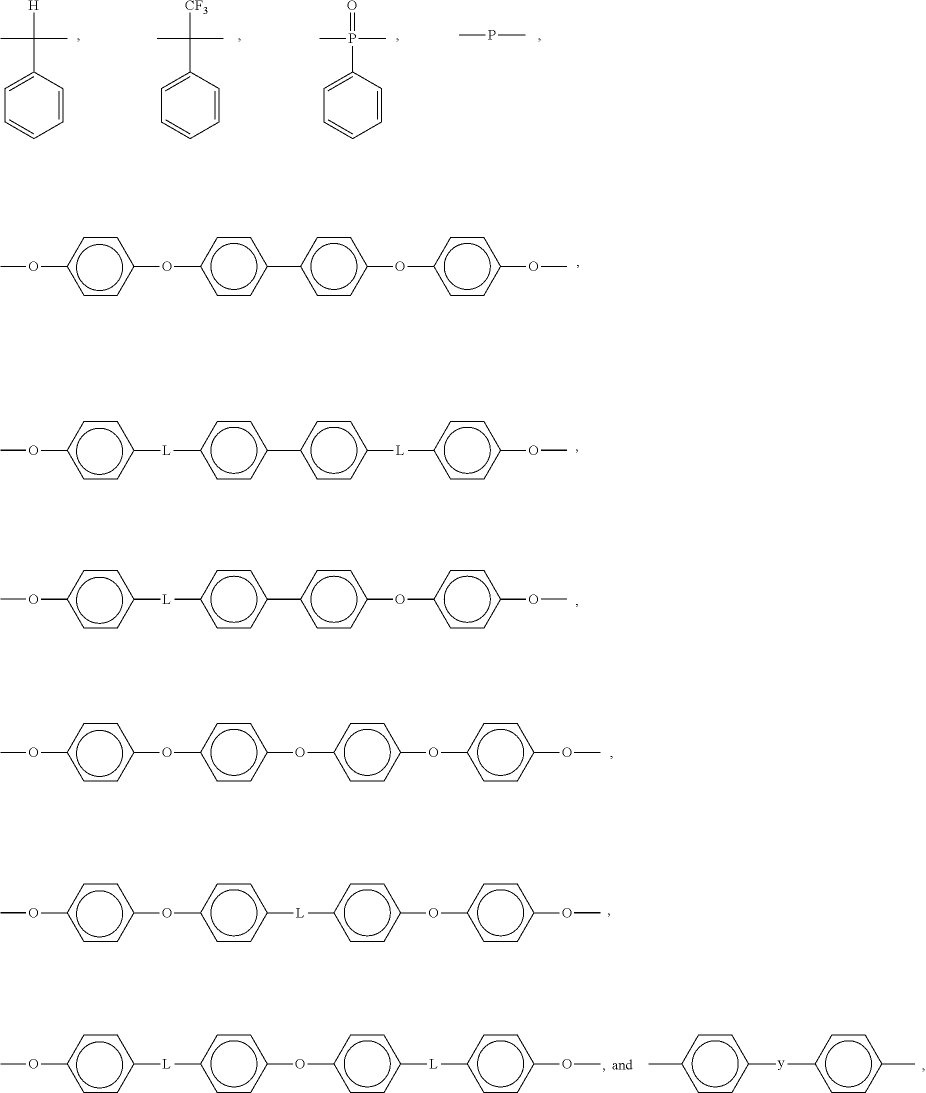
wherein L is —CH₂—, —(CH₃)₂C—, —S—, —SO₂—, or —CO—;
wherein y is —SO₂—, —S—, —(CF₃)₂C—, —O—, or —(CH₃)₂C—;
and n is selected such that the molecular weight of the polyimide oligomer is between 1000 and 3000; and
a reinforcement material.

2. The composite according to claim 1, wherein the dinadic phenyl amine endcap is:

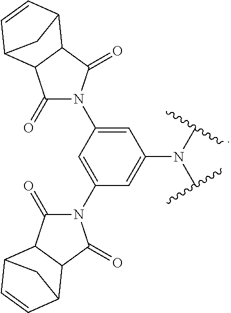 [SMILES: *N(*)c1cc(N2C(=O)C3C4C=CC(C4)C3C2=O)cc(N2C(=O)C3C4C=CC(C4)C3C2=O)c1]

3. The composite according to claim 1, wherein the dinadic phenyl amine endcap is:

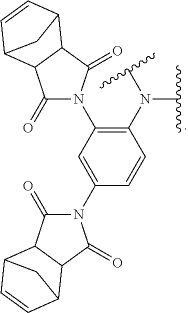 [SMILES: *N(*)c1ccc(N2C(=O)C3C4C=CC(C4)C3C2=O)cc1N1C(=O)C2C3C=CC(C3)C2C1=O]

4. The composite according to claim 1, wherein the dinadic phenyl amine endcap is:

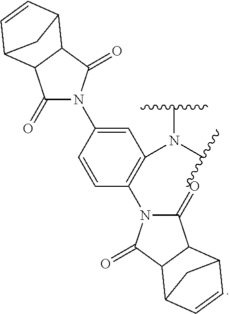

5. A composite comprising:
polyimide oligomer of Formula: 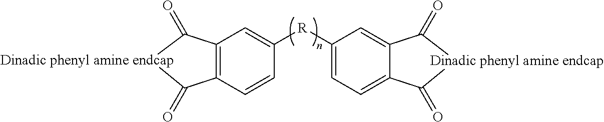
wherein each dinadic phenyl amine endcap is selected from the group consisting of 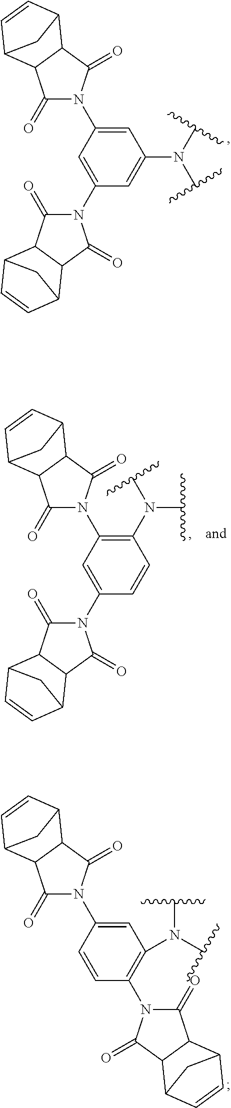
wherein R is selected from the group consisting of 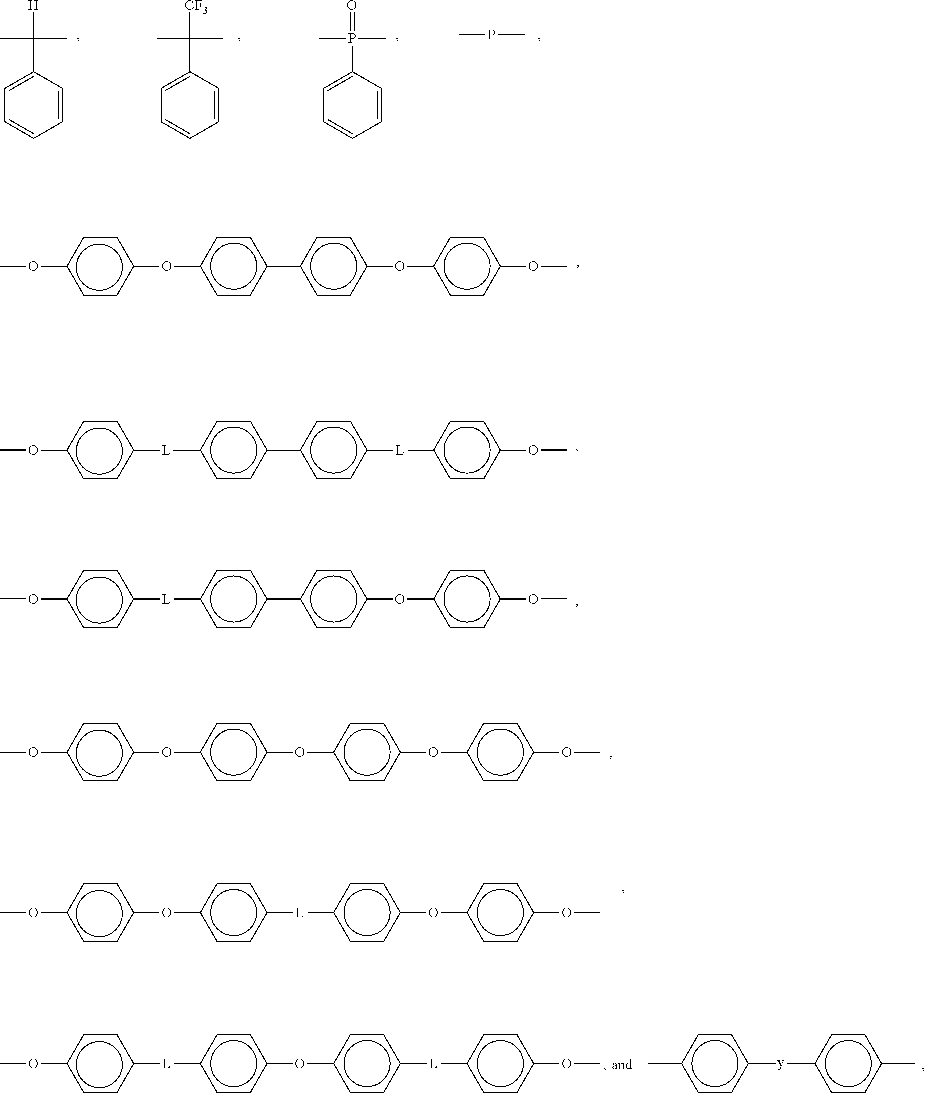
wherein L is —CH₂—, —(CH₃)₂C—, —S—, —SO₂—, or —CO—;
wherein y is —SO₂—, —S—, —(CF₃)₂C—, —O—, or —(CH₃)₂C—;
wherein n is selected such that the molecular weight of the polyimide oligomer is less than 5000; and
a reinforcing material.

6. The composite according to claim 5, wherein the dinadic phenyl amine endcap is:

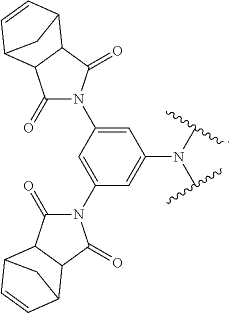 [SMILES: *N(*)c1cc(N2C(=O)C3C4C=CC(C4)C3C2=O)cc(N2C(=O)C3C4C=CC(C4)C3C2=O)c1]

7. The composite according to claim 5, wherein the dinadic phenyl amine endcap is:

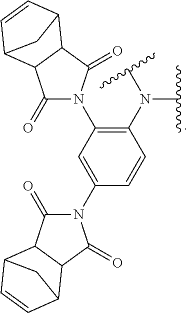 [SMILES: *N(*)c1ccc(N2C(=O)C3C4C=CC(C4)C3C2=O)cc1N1C(=O)C2C3C=CC(C3)C2C1=O]

8. The composite according to claim 5, wherein the dinadic phenyl amine endcap is:

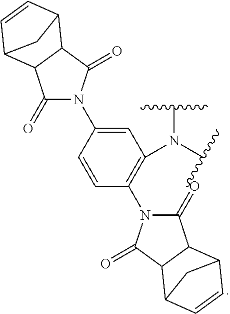

9. The composite according to claim 5, wherein R is selected from the group consisting of 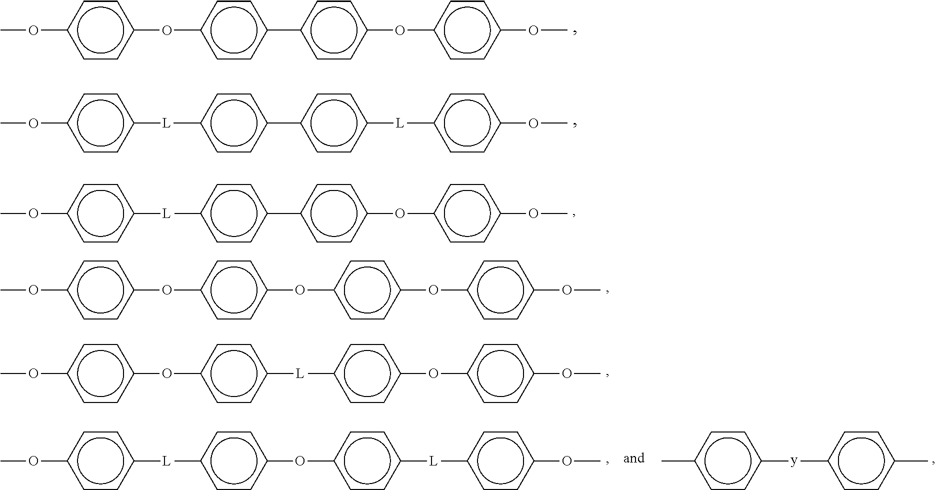
wherein L is —CH₂—, —(CH₃)₂C—, —S—, —SO₂—, or —CO—;
wherein y is —SO₂—, —S—, —(CF₃)₂C—, —O—, or —(CH₃)₂C—;

10. The composite according to claim 5, wherein R is selected from the group consisting of

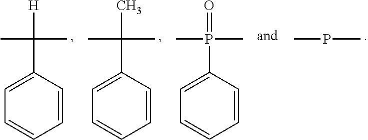

11. The composite according to claim 10, wherein n is selected such that the molecular weight of the polyimide oligomer is between 1000 and 3000.

12. The composite according to claim 10, wherein L and y are —SO₂—.

13. The composite according to claim 9, wherein L and y are —SO₂—.

14. A composite comprising:
polyimide oligomer of Formula: 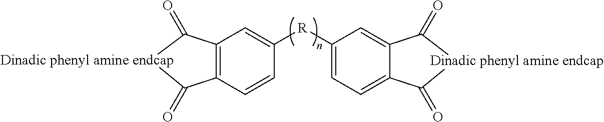
wherein each dinadic phenyl amine endcap is selected from the group consisting of: 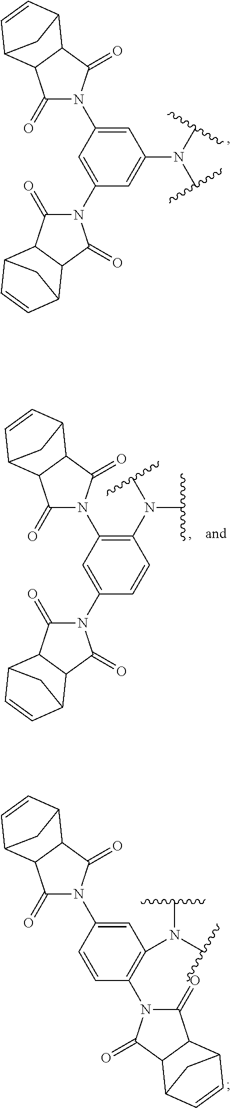
wherein R is selected from the group consisting of 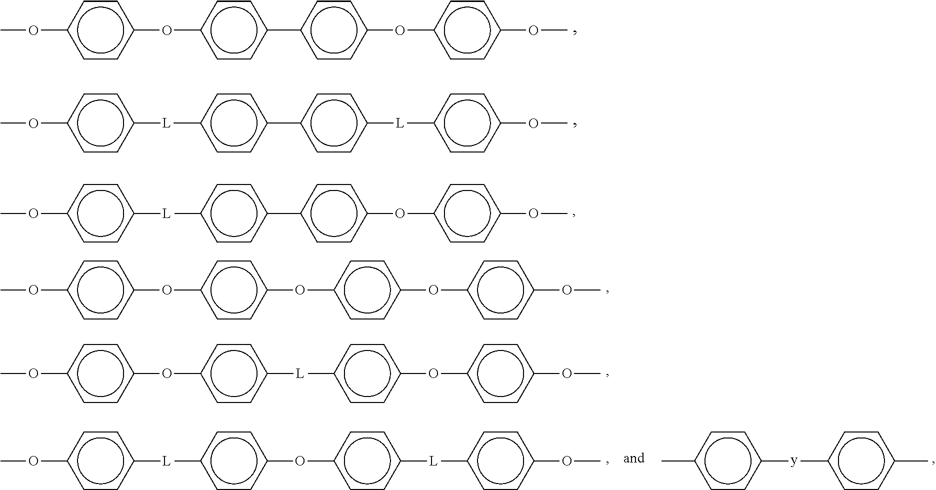
wherein L is —CH₂—, —(CH₃)₂C—, —S—, —SO₂—, or —CO—;
wherein y is —SO₂—, —S—, —(CF₃)₂C—, —O—, or —(CH₃)₂C—;
wherein n is selected such that the molecular weight of the polyimide oligomer is between 1000 and 3000; and
a reinforcing material.

15. The composite according to claim 14, wherein L is —SO₂— and y is —SO₂—.

16. The composite according to claim 14, wherein R is selected from the group consisting of

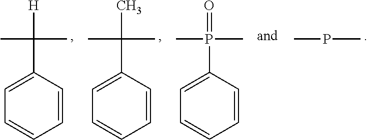

17. The composite according to claim 16, wherein L is —SO₂— and y is —SO₂—.

18. The composite according to claim 1, wherein the reinforcement material is selected from a woven fabric, a continuous or discontinuous fiber, a ceramic, an organic, a carbon material, or a glass.

19. The composite according to claim 5, wherein the reinforcement material is selected from a woven fabric, a continuous or discontinuous fiber, a ceramic, an organic, a carbon material, or a glass.

20. The composite according to claim 14, wherein the reinforcement material is selected from a woven fabric, a continuous or discontinuous fiber, a ceramic, an organic, a carbon material, or a glass.
